(19) United States
(12) Patent Application Publication
(10) Pub. No.: US 2006/0117492 A9
(43) Pub. Date: Jun. 8, 2006

(54) Composition comprising at least one conductive polymer and at least one reducing agent, and permanent-shaping process for the use thereof

(76) Inventors: Isabelle Rollat-Corvol, Paris (FR); Henri Samain, Bievres (FR)

Correspondence Address: Thomas L. Irving;FINNEGAN, HENDERSON, FARABOW,, GARRETT & DUNNER, L.L.P., 1300 I Street, N.W., Washington, DC, 20005-3315, US

(21) Appl. No.: 10/892,336

(22) Filed: Jul. 16, 2004

Related U.S. Application Data
(60) Provisional application No. 60/492,289, filed on Aug. 5, 2003.

(30) Foreign Application Priority Data
Jul. 16, 2003 (FR) 03 08674

(51) Int. Cl. A61K 7/13

(52) U.S. Cl. 8/405

(57) ABSTRACT

A composition comprising, in a cosmetically acceptable medium, (a) at least one reducing agent and (b) at least one conductive polymer. A process for treating keratin fibers, for example, human keratin fibers, such as hair, comprising a) applying, to wet or dry keratin fibers, the at least one composition disclosed herein; b) leaving the at least one composition on the fibers for a time period that is sufficient to shape the fibers; c) optionally rinsing the fibers and optionally applying at least one oxidizing composition to the keratin fibers; d) leaving the at least one oxidizing composition on the keratin fibers for a time period that is sufficient to fix the shape of the fibers; e) optionally rinsing the fibers; f) washing and rinsing the fibers; and g) drying the fibers or leaving the fibers to dry. The use of the composition for imparting an optical effect on keratin fibers.

This application claims benefit of U.S. Provisional Application No. 60/492,289, filed Aug. 5, 2003.

Disclosed herein is a composition comprising, in a cosmetically acceptable medium, at least one conductive polymer and at least one reducing agent. Further disclosed herein is a process for treating keratin fibers, for example, permanently reshaping keratin fibers, using the abovementioned composition. Also, disclosed herein is use of such a composition to impart at least one optical effect on keratin fibers.

Keratin fibers, for example, human keratin fibers, such as hair, may be subjected to various treatments, including permanent shaping.

Permanent shaping may comprise two steps. The first step comprising placing fibers in contact with a reducing composition in order to reduce the disulfide bridges present in the keratin fiber. Before, after or simultaneously with the reduction of these disulfide bridges, the fibers may be shaped in the desired manner. Shaping may comprise curling the hair or smoothing it out, the result depending on the means used to place the hair under tension. Once this first step of reduction and of placing under tension has been performed, a fixing step may be necessary to re-establish the disulfide bridges and thus stabilize the given shape. This operation may usually be performed in an oxidizing medium.

It is clear that this type of process may be relatively aggressive towards the keratin fiber, and after such a treatment the fiber may be damaged and have a somewhat dull appearance.

Moreover, it is common to need to perform permanent-shaping and dyeing steps with a very small time interval between them, or even immediately one after the other, the shaping operation being performed first.

In this case also, it may be clearly seen that performing a subsequent dyeing step represents an additional risk of degradation, because the dyeing processes may often be performed in the presence of an oxidizing agent, in an alkaline medium.

It is still possible to treat the fibers with an agent that provides them, for example, with sheen; it being possible for the agent either to be present in the dye composition itself, or to be provided in a composition applied after the dyeing treatment.

However, the sheen effect obtained may lack intensity and may give the fibers an artificial appearance.

In addition, such compositions may have the drawback of giving the fibers a greasy or tacky feel.

Finally, if one wishes to perform a dyeing step after the shaping process, the presence of the abovementioned agents may limit the uptake of the dye into the fibers and consequently give less intense colorations.

One aim of the present inventors is thus to propose compositions comprising at least one reducing agent and at least one conductive polymer, which may give treated keratin fibers a particular optical effect without at least some of the drawbacks encountered with prior art compositions.

Moreover, when the at least one conductive polymer absorbs in the range of the visible spectrum, the composition disclosed herein may make it possible to dye the fibers without it being necessary to perform a particular dyeing treatment subsequent to the treatment for shaping the fibers.

Finally, the keratin fibers may have a soft, pleasant feel after the composition disclosed herein is applied.

Disclosed herein is thus a composition comprising, in a cosmetically acceptable medium:
(a) at least one reducing agent and
(b) at least one conductive polymer.

Further disclosed herein is a process for treating keratin fibers, for example, human keratin fibers, such as hair, comprising:
a) applying at least one composition comprising, in a cosmetically acceptable medium,
   (i) at least one reducing agent and
   (ii) at least one conductive polymer;
b) leaving the at least one composition on the fibers for a time period that is sufficient to shape the fibers;
c) optionally rinsing the fibers and optionally applying at least one oxidizing composition to the keratin fibers;
d) leaving the at least one oxidizing composition on the keratin fibers for a time period that is sufficient to fix the shape of the fibers;
e) optionally rinsing the fibers;
f) washing and rinsing the fibers; and
g) drying the fibers or leaving the fibers to dry.

Even further disclosed herein is the use of a composition comprising at least one reducing agent and at least one conductive polymer, to give keratin fibers at least one optical effect.

Specifically, in one embodiment, the composition disclosed herein gives the keratin fibers a particular optical effect, for example, sheen that may be at least one of substantially more intense, more natural and more aesthetic than with the compositions of the prior art.

Moreover, when the at least one conductive polymer present in the composition disclosed herein absorbs in the visible spectrum, at least one optical effect, for example, chosen from sheen and color may be obtained simultaneously.

In one embodiment, it may no longer be necessary to perform a subsequent dyeing process.

In the text hereinbelow and unless otherwise indicated, the limits of a range of values are understood as forming part of that range.

As used herein, the term “optical effect” means sheen, color, metallic, goniochromatic and moiré effects.

Moreover, it should be noted that, as used herein, “sheen” corresponds to the light intensity reflected at an angle α when a lock of hair is illuminated under an angle −α. The angle α conventionally used to measure this specular reflection, in other words the sheen, is equal to 20°. This provision of sheen is measured using a glossmeter as described in ISO standard 2813-1994 from AFNOR (August 1994, amended February 1997).

Conductive Polymers

As used herein, the term “conductive polymer” means a molecular structure in which the monomer(s) has (have) high electron delocalization and whose arrangement in the polymer skeleton allows the it orbitals to overlap. This chemical characteristic is reflected by electrical conduction, which may or may not be accompanied by absorption in the UV-visible spectrum, or even in the infrared spectrum.

As used herein, the expression “conductive polymer absorbing in the visible spectrum” means any conductive polymer having a non-zero absorbance in the wavelength ranging from 400 to 800 nm, even if the absorption maxima of the polymer is outside this range.

The at least one conductive polymer used in the composition disclosed herein is chosen from conductive polymers that are soluble or dispersible in the cosmetic medium suitable for use.

The at least one conductive polymer is soluble in the medium when it forms an isotropic clear liquid at 25° C. in a medium chosen from water and mixtures of water and at least one solvent, wherein the solubility is obtained throughout all or part of a concentration ranging from 0.01% to 50% by weight of the at least one conductive polymer.

In one embodiment, the at least one conductive polymer is chosen from conductive polymers that are soluble or dispersible in an aqueous medium, for example, in water.

The at least one conductive polymer is dispersible in the medium comprising water or mixtures of water and at least one solvent if, at 0.01% by weight, at 25° C., it forms a stable suspension of fine, generally spherical particles. The mean size of the particles constituting the dispersion is less than 1 μm, for example, ranging from 5 to 400 nm and, further, for example, from 10 to 250 nm. These particle sizes are measured by light scattering.

It should be noted, that in some embodiments, the at least one conductive polymer does not require the use of a dispersant.

The at least one conductive polymer may, for example, be in a form that is soluble in the medium of the composition.

Further, the at least one conductive polymer may have a conductivity ranging from 1×10⁻⁵ to 5×10⁵ siemens/cm, further, for example, from 1×10⁻³ to 1×10⁵ siemens/cm and, even further, for example, from 1×10⁻¹ to 1×10⁴ siemens/cm.

The conductivity is measured using a current generator (RM2 Test Unit sold by the company Jandel) equipped with a four-point measuring head (Universal four-point probes sold by the company Jandel). The four points, aligned and separated by the same space d, are applied by simple pressure to the sample to be analysed. A current I is injected via the outer points using the current source, thus creating a variation in potential. The voltage U is measured between the two inner points connected to the voltmeter of the current generator.

In this configuration, the conductivity of the sample expressed in S/cm is given by the following expression:
σ=(K×I)/(U×e)
wherein:

K is a coefficient depending on the position of the contacts on the surface of the sample. When the points are aligned and equidistant, K is equal to: π/log(2);
I is the value of the injected current, expressed in amperes;
U is the measured voltage value, expressed in volts; and
e is the thickness of the sample, expressed in cm.

This expression can be used only when the thickness of the material is negligible compared with the distance d existing between two points (e/d<0.25). In order to obtain a sufficiently small thicknesses and thus to be able to calculate the conductivity of the material, it is recommended to perform the measurement on a non-conductive support (for example a glass slide) coated with the material to be analysed, obtained by evaporation of a dilute solution. In order to improve the homogeneity of the coating to be analysed, it is also recommended to use the deposition technique known as spin coating.

In one embodiment, the at least one conductive polymer present in the composition disclosed herein is chosen from polymers comprising at least one repeating unit of the following formulae:
anilines of formula (I) below: 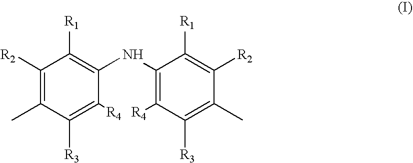
pyrroles of formulae (IIa) and (IIb) below: 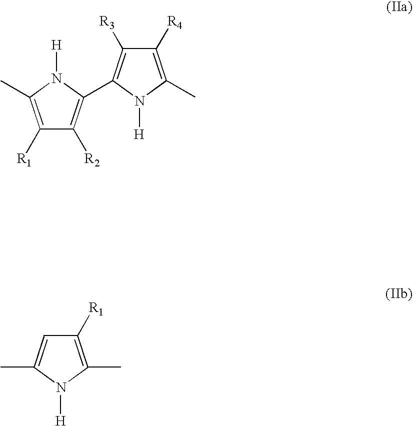
thiophenes and bisthiophenes of formulae (IIIa), (IIIb) and (IIIc) below: 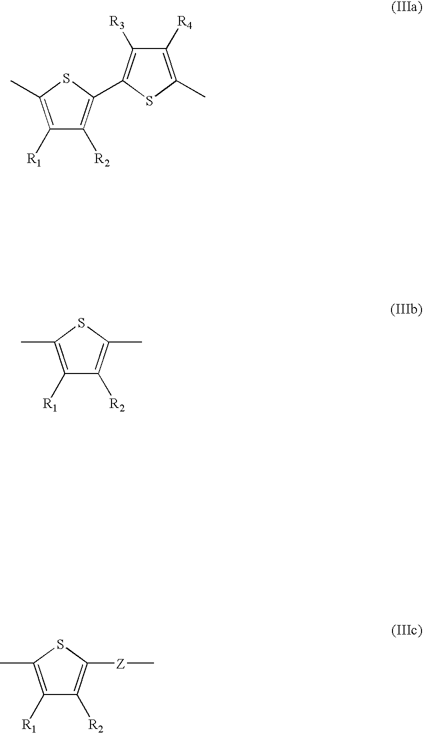
furans of formula (IV) below: 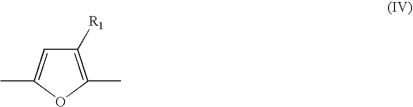
para-phenylene sulfides of formula (V) below: 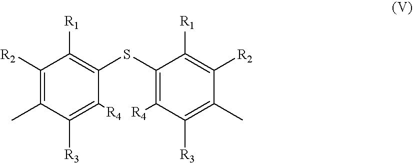
para-phenylenevinylenes of formula (VI) below: 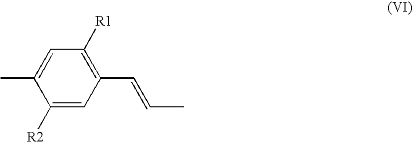
indoles of formula (VII) below: 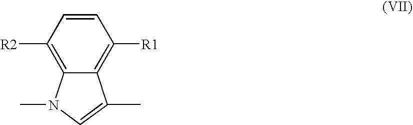
aromatic amides of formulae (VIIIa), (VIIIb), (VIIIc) and (VIIId) below 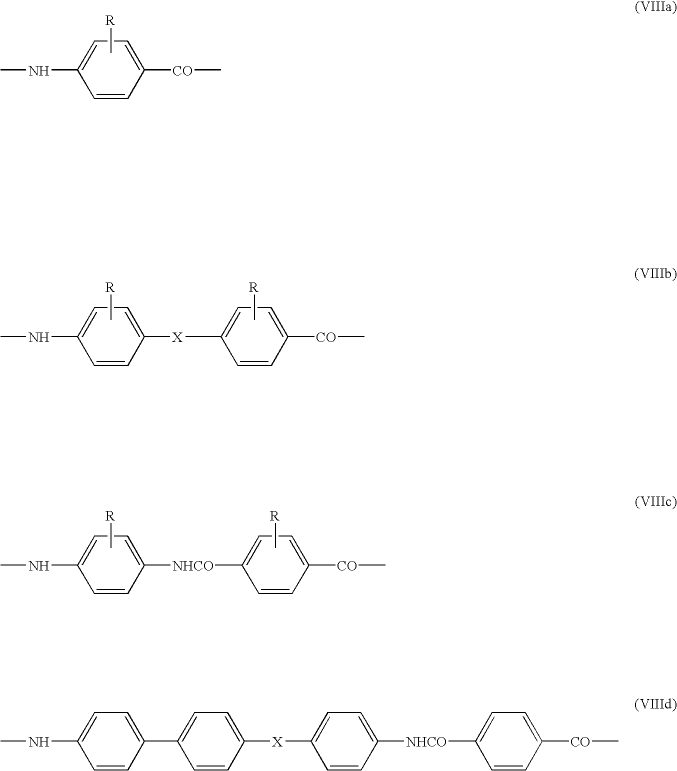
aromatic hydrazides of formulae (IXa), (IXb) and (IXc) below: 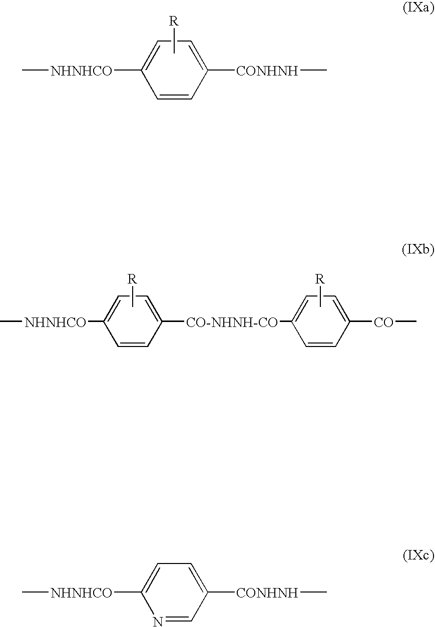
aromatic azomethines of formulae (Xa), (Xb) and (Xc) below: 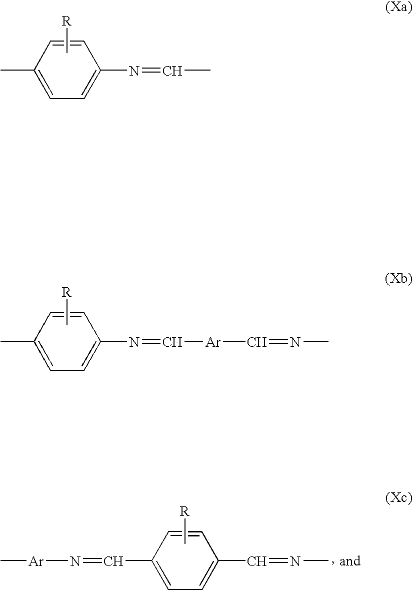
aromatic esters of formulae (XIa), (XIb) and (XIc) below: 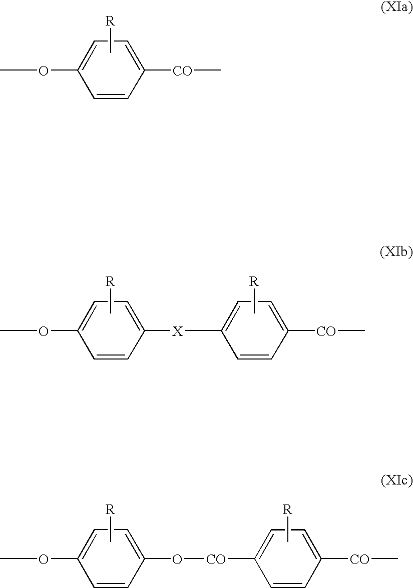
   wherein, in formulae (I) to (XI):
radicals R, R₁, R₂, R₃, and R₄, which may be identical or different, are each chosen from a hydrogen atom; radicals —R′, —OR′, —COOR′, and —OCOR′, wherein R′ is chosen from linear and branched C₁-C₂₀ alkyl radicals; halogen atoms; nitro radicals; cyano radicals; cyanoalkyl radicals; solubilizing groups; and solubilizing groups comprising a spacer group that bonds to the ring;
Ar is chosen from radicals comprising at least one radical chosen from monoaromatic and polyaromatic radicals;
X is chosen from —NHCO—, —O—, —S—, —SO₂—, —N═N—, —C(CH₃)₂—, —CH₂—, —CH═CH—, and —CH═N—; and
Z is chosen from —CH═CH— and —C≡C—.

In one embodiment, Ar is chosen from radicals comprising at least one radical chosen from:

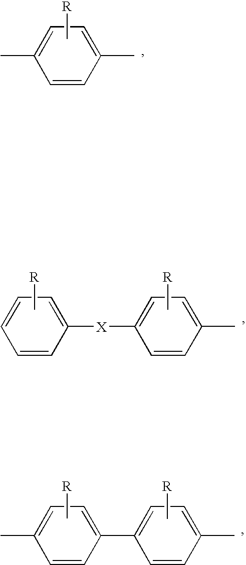

As used herein, the term “solubilizing group” means a group that ensures the dissolution of the molecule in the cosmetic medium, such that the at least one conductive polymer has a conductive nature after drying the composition.

It is clear that the at least one conductive polymer present in the composition disclosed herein may comprise at least one repeating unit comprising at least one solubilizing group, and at least one other repeating unit lacking the at least one solubilizing group.

The solubilizing groups may, for example, be chosen from the following radicals:
carboxylic (—COOH) radicals and carboxylate (—COO⁻M⁺) radicals, wherein M is chosen from alkali metals, such as sodium and potassium; alkaline-earth metals; organic amines, such as a primary, secondary and tertiary amine; alkanolamines; and amino acids;
sulfonic (—SO₃H) radicals and sulfonate (—SO₃⁻M⁺) radicals, wherein M has the same definition as above;
primary, secondary, and tertiary amine radicals;
quaternary ammonium radicals, such as —N(R′)₃⁺Z⁻ wherein Z is chosen from Br, Cl, and (C₁-C₄)alkyl-OSO₃ and R′, which may be identical or different, is chosen from linear and branched C₁ to C₂₀ alkyls, or two of the R's may form a heterocycle with the nitrogen to which they are attached;
hydroxyl radicals; and
poly((C₂-C₃)alkylene oxide) radicals.

The carboxylic or sulfonic acid radicals may be optionally neutralized with at least one base, for example, chosen from sodium hydroxide, 2-amino-2-methylpropanol, triethylamine, and tributylamine.

The amine radicals may be optionally neutralized with at least one mineral acid, for example, chosen from hydrochloric acid and organic acids, such as acetic acid and lactic acid.

In addition, it should be noted that the solubilizing groups may be connected to the ring via a spacer group, for example, chosen from radicals —R″—, —OR″—, —OCOR″—, and —COOR″— wherein R″ is chosen from linear and branched C₁-C₂₀ alkyl radicals optionally comprising at least one hetero atom, for example, oxygen.

In one embodiment, the radicals R and R₁ to R₄, which may be identical or different, are each chosen from hydrogen, R′, —OR′, —OCOR′, and —COOR′ wherein R′ is chosen from linear and branched C₁-C₆ alkyl radicals, and from the following neutralized or non-neutralized solubilizing groups: —COOH, —CH₂COOH, —CH₂OH, —(CH₂)₆OH, —(CH₂)₃SO₃H, —O(CH₂)₃SO₃H, —O(CH₂)₃N(CH₂CH₃)₂, —[(CH₂)₂O]ₓCH₂CH₂OH, and —[(CH₂)₂O]ₓCH₂CH₂OCH₃, wherein x is an average number ranging from 0 to 200.

The number n of repeating units in the at least one conductive polymer may, for example, range from 5 to 10 000, for example, from 5 to 1000, further, for example, from 10 to 1000 and, even further, for example, from 20 to 700.

In one embodiment, the at least one conductive polymer is such that at least one radical chosen from R, R₁, R₂, R₃, and R₄, is a solubilizing group.

In yet another embodiment, the at least one conductive polymer comprises at least one solubilizing group per repeating unit. Thus, for example, the at least one radical chosen from R, R₁, R₂, R₃, and R₄ is a solubilizing group.

In another embodiment, the at least one conductive polymer is soluble in the medium of the composition.

The at least one conductive polymer present in the composition disclosed herein may be well known to those skilled in the art and may be described, for example, in the book “Handbook of Organic Conductive Molecules and Polymers”—Wiley 1997—New York, Vol 1, 2, 3, and also in the review Can. J. Chem. Vol 64,1986.

Polythiophenes and their synthesis are, for example, described in the article taken from the review Chem. Mater. 1998, Vol. 10, No 7, pages 1990-1999 by the authors Rasmussen S. C., Pickens J. C. and Hutchison J. E. “A New, General Approach to Tuning the Properties of Functionalized Polythiophenes: The Oxidative Polymerization of Monosubstituted Bithiophenes;” in the article taken from the review Macromolecules 1998, 31, pages 933-936, by the same authors “Highly Conjugated, Water-Soluble Polymers Via Direct Oxidative Polymerization of Monosubstituted Bithiophenes”. In addition to polymerization via chemical or electrochemical oxidation, the polythiophenes may also be obtained by at least one reaction chosen from polycondensation (dihalothiophene; catalysis with nickel or palladium complexes); Suzuki coupling (coupling between a halogen functional group, for example bromine, and a boronic acid, catalysis: palladium complex and base; this then gives coupling of AA-BB type (reaction of monomers of the type A-X-A with B-X′-B) or of A-B type (reaction of several monomers of the type A-X-B); Stille coupling (formation of a carbon-carbon bond in the presence of a Pd-based catalyst—AA-BB or A-B type); Reike polymerization (organozinc in the presence of a nickel complex); and polymerization of McCulloch type, etc.

The at least one conductive polymer present in the composition disclosed herein is moreover described in International Patent Publication No. WO 99/47570.

Examples of the at least one conductive polymer that are suitable for use in the composition disclosed herein include the polymers comprising at least one repeating unit chosen from units corresponding to formulae (IIIa), (IIIb) and (IIIc) wherein the solubilizing groups may, for example, be chosen from carboxylic acid radicals; neutralized carboxylic acid radicals, sulfonic acid radicals; neutralized sulfonic acid radicals; tertiary amine radicals; and quaternary ammonium radicals, such as —NR₁₃+Z-, wherein Z is chosen from Br, Cl, (C₁-C₄)alkyl-OSO₃ and R′, which may be identical or different, is chosen from linear and branched C₁ to C₂₀ alkyls, or two of the R's form a heterocycle with the nitrogen to which they are attached; wherein the groups are optionally connected to the ring via a spacer group.

Thus, the polymerization may be performed via chemical or electrochemical oxidation of the corresponding thiophene monomer or via polycondensation.

For example, the polythiophenes of formulae (IIIa) and (IIIb) may be obtained by at least one reaction chosen from the following:
polymerization via oxidation (for example, with FeCl₃ catalysis);
polycondensation of dihalothiophene catalysed with nickel or palladium complexes (e.g.: NiCl₂(dppe)₂);
Suzuki coupling (coupling between a halogen functional group, for example, bromine, and a boronic acid, catalysis: palladium complex and base; this then gives coupling of AA-BB type (reaction of monomers of the type A-X-A with B-X′-B) and of A-B type (reaction of several monomers of the type A-X-B);
Stille coupling (formation of a carbon-carbon bond formed in the presence of a Pd-based catalyst—AA-BB or A-B type);
Reike polymerization (organozinc in the presence of a nickel complex); and
polymerization of McCulloch type, etc.

The vinylene polythiophenes of formula (IIIc) wherein Z is a —CH═CH— radical may be obtained by at least one reaction chosen, for example, from Gilch polymerization in the presence of a strong base (potassium tert-butoxide) of 2,5-bis(bromoalkylene)thiophene; polymerization by the Wessling method via the use of a precursor based on sulfonium salts and pyrolysis; and a Wittig-Horner Wittig reaction.

The ethynylene polythiophenes of formula (IIIc) wherein Z is a —C≡C— radical may be obtained by at least one reaction chosen from Heck-Sonogashira coupling (of the type AA-BB or A-B; formation of a carbon-carbon bond between a terminal acetylenic (or true acetylenic) functional group and a bromo or iodo functional group, catalysed with a palladium/copper complex (PdCl₂(PPh₃)₃, Cul or Cu(OAc)₂) in the presence of a base such as triethylamine, diisopropyl amine, piperidine, etc.); and metathesis of alkynes in the presence of a molybdenum complex (Mo(CO)₆).

In general, the functionalization of the polythiophenes, in other words the introduction of the solubilizing or non-solubilizing group(s), is performed on the monomer before it is polymerized.

In some embodiments, the solubilizing group may be obtained after working up the polymer. This may, for example, be the case for the carboxylic acid functional group, which may be obtained by hydrolysis of the corresponding ester.

For example, in one embodiment, the solubilizing groups are chosen from carboxylic acid radicals; neutralized carboxylic acid radicals; sulfonic acid radicals; neutralized sulfonic acid radicals; tertiary amine radicals; quaternary ammonium radicals, such as —N(R′)₃⁺Z⁻, wherein Z is chosen from Br, Cl, and (C₁-C₄)alkyl-OSO₃ and R′, which may be identical or different, is chosen from linear and branched C₁-C₂₀ alkyl radicals, optionally connected to the ring via a spacer group, for example, chosen from C₁-C₂₀ alkyl radicals; and salts thereof.

In another embodiment, the at least one conductive polymer comprises at least one repeating unit chosen from units of formulae (IIIa), (IIIb) and (IIIc), wherein at least one radical chosen from R₁, R₂, R₃, and R₄ of formula (IIIa) and R₁ or R₂ of formulae (IIIb) and (IIIc) is chosen from carboxylic acid solubilizing radicals, in neutralized or non-neutralized form, optionally connected to the ring via a spacer group, for example, chosen from linear and branched C₁-C₂₀ alkyl radicals, wherein the other radical(s) is (are) hydrogen(s).

The at least one conductive polymer may be present in the composition in amount greater than or equal to 0.001% by weight, for example, greater than or equal to 0.01% by weight, further, for example, greater than or equal to 0.1% by weight, and even further, for example, greater than or equal to 0.5% by weight, relative to the total weight of the composition. Moreover, the at least one conductive polymer may be present in an amount less than or equal to 50% by weight, for example, less than or equal to 30% by weight, further, for example, less than or equal to 20% by weight, and, even further, for example, less than or equal to 10% by weight, relative to the total weight of the composition.

In yet another embodiment, the at least one conductive polymer is present in an amount ranging from 0.1% to 50% by weight, for example, from 0.1% to 30% by weight, and, even further, for example, from 0.5% to 10% by weight, relative to the total weight of the composition.

Reducing Agents

As mentioned above, the composition disclosed herein comprises, besides the at least one conductive polymer, at least one reducing agent.

The at least one reducing agent may be chosen from sulfur-comprising reducing agents and reductones.

In one embodiment, the sulfur-comprising reducing agents are chosen from compounds comprising at least one functional group chosen from thiol, sulfide, and sulfite functional groups.

Examples of sulfur-comprising reducing agents include thioglycolic acid, P-mercaptoethanol, thiolactic acid, alkali metal and alkaline-earth metal salts thereof and esters thereof; cysteine, cysteamine, and derivatives thereof; homocysteine and a salt thereof; mercaptoaldehyde; penicillamine; glutathione; thioglycolate; alkali metal, alkaline-earth metal and ammonium sulfites, bisulfites and hydrosulfites; cystine; and mixtures thereof.

Among the alkali metals, sodium and potassium may be used, and among the alkaline-earth metals, calcium may, for example, be used. With regard to the ammonium groups, they may be optionally substituted with at least one alkyl radical chosen from linear and branched alkyl radicals comprising from 1 to 4 carbon atoms, optionally bearing at least one hydroxyl group.

The reductones may be chosen, for example, from ascorbic acid and isoascorbic acid, and salts and esters thereof; hydroxypropanedial; 2,3-hydroxy-2-cyclopenten-1-one; and α-ketoglutaric acid.

The at least one reducing agent may be present in an amount ranging from 1% to 30% by weight and, for example, from 5% to 20% by weight, relative to the total weight of the composition.

Conventionally, the cosmetically acceptable medium of the composition is chosen from water and mixtures of water and at least one cosmetically acceptable organic solvent.

The at least one cosmetically acceptable organic solvent may be chosen from C₁-C₄ alcohols, such as ethyl alcohol and isopropyl alcohol; aromatic alcohols, for example, benzyl alcohol and phenylethyl alcohol; glycols and glycol ethers, such as ethylene glycol monomethyl ether, monoethyl ether and monobutyl ether, propylene glycol and ethers thereof, such as propylene glycol monomethyl ether, butylene glycol, dipropylene glycol and diethylene glycol alkyl ethers, for example, diethylene glycol monoethyl ether and monobutyl ether; and polyols, such as glycerol. Polyethylene glycols and polypropylene glycols, and mixtures of all these compounds, may also be used as the at least one cosmetically acceptable organic solvent.

If present, the at least one cosmetically acceptable organic solvent described above may be present in an amount ranging from 1% to 40% by weight, and, for example, from 5% to 30% by weight, relative to the total weight of the composition.

The reducing composition may also comprise at least one common surfactant. The at least one surfactant may be chosen from nonionic, anionic, cationic, amphoteric, and zwitterionic surfactants. Examples of the at least one surfactant include alkyl sulfates, alkylbenzene sulfates, alkyl ether sulfates, alkyl sulfonates, quaternary ammonium salts, alkylbetaines, oxyethylenated alkylphenols, fatty acid alkanolamides, oxyethylenated fatty acid esters, and hydroxypropyl ether nonionic surfactants.

When the reducing composition comprises this type of surfactant, the at least one surfactant may be present is amount less than or equal to 30% by weight and, for example, ranging from 0.5% to 10% by weight, relative to the total weight of the composition.

The pH of the composition may range, for example, from 3 to 12, further, for example, from 5 to 11.

It may be adjusted to the desired value using acidifying or basifying agents.

Examples of acidifying agents include mineral and organic acids, such as hydrochloric acid, orthophosphoric acid, and sulfuric acid; carboxylic acids, such as acetic acid, tartaric acid, citric acid and lactic acid; and sulfonic acids.

Examples of alkaline agents include aqueous ammonia, alkaline carbonates, alkanolamines, such as monoethanolamine, diethanolamine and triethanolamine, and derivatives thereof, sodium hydroxide, potassium hydroxide and compounds of formula (A) below: 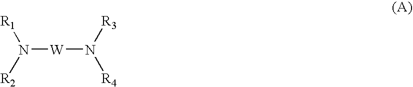
wherein W is a propylene residue optionally substituted with at least one entity chosen from hydroxyl groups and C₁-C₆ alkyl radicals; R₁, R₂, R₃ and R₄, which may be identical or different, are each chosen from a hydrogen atom and C₁-C₆ alkyl and C₁-C₆ hydroxyalkyl radicals.

The composition may also comprise at least one conventionally used adjuvant, for example, chosen from anionic, cationic, nonionic, amphoteric, and zwitterionic polymers; organic and mineral thickeners; nonionic, anionic, and amphoteric associative polymers; antioxidants; penetrating agents; sequestering agents; fragrances; buffers; dispersants; conditioners; film-forming agents; ceramides; preserving agents; stabilizers; and opacifiers.

When the compositions are intended for a smoothing operation, for example, relaxing or straightening the hair, the reducing composition may, for example, be in the form of a thickened cream so as to keep the hair as straight as possible during this step. These creams are made in the form of “heavy” emulsions, obtained, for example, by emulsifying an aqueous phase, for example, an aqueous phase comprising the at least one reducing agent and the at least one conductive polymer, and an oily phase comprising, for example, at least one component chosen from plant oil, liquid paraffin, fatty acid esters, and waxes.

It is also possible to use liquids or gels comprising thickeners, such as carboxyvinyl polymers or copolymers, which “stick” the hairs together and keep them in the smooth position during the leave-in time.

If the operation is intended to make the fibers curly, these fibers are placed under tension using curlers, before, during or after the application of the composition disclosed herein.

The leave-in time period may range from 3 to 30 minutes and, for example, from 5 to 15 minutes.

The oxidizing composition used during step c) in the treatment process disclosed herein conventionally comprises at least one oxidizing agent, for example, chosen from aqueous hydrogen peroxide solution, an alkaline bromate, a persalt, and a polythionate.

The at least one oxidizing agent may be present in an amount ranging from 0.1% to 25% by weight, relative to the weight of the oxidizing composition.

The oxidizing composition may also comprise at least one surfactant, for example, chosen from nonionic, anionic and amphoteric compounds. Examples of the at least one surfactant include alkyl sulfates, alkyl ether sulfates, betaines, imidazolium derivatives, alkylpyrrolidones, oxyalkylenated and glycerolated fatty alkyl ethers, and optionally oxyalkylenated and glycerolated fatty acid esters of monoalcohols and of polyols.

If present, the at least one surfactant may be present in an amount ranging from 0.01% to 30% by weight, relative to the total weight of the oxidizing composition, further, for example, ranging from 0.1% to 20% by weight, and, even further, for example, ranging from 0.2% to 10% by weight, relative to the total weight of the oxidizing composition.

The oxidizing composition may also comprise at least one thickener, and, for example, thickening systems based on associative polymers that are well known to those skilled in the art, for example, chosen from nonionic, anionic, cationic, and amphoteric associative polymers. Examples of these polymers include crosslinked acrylic acid homopolymers; partially and totally neutralized crosslinked homopolymers and copolymers of 2-acrylamido-2-methylpropanesulfonic acid; ammonium acrylate homopolymers and copolymers; quaternized dimethylaminoethyl methacrylate homopolymers and copolymers; nonionic guar gums, biopolysaccharides of microbial origin (scleroglucan and xanthan), derived from plant exudates (gum arabic, ghatti gum, karaya gum, and gum tragacanth); hydroxypropyl- and carboxymethylcelluloses; pectins; and alginates.

If present, the at least one thickener may be present in an amount ranging from 0.01% to 10% by weight, and, for example, from 0.1% to 5% by weight, relative to the total weight of the oxidizing composition.

It should moreover be noted that the oxidizing composition may comprise at least one conditioner, for example, chosen from cations, volatile and non-volatile, modified and unmodified silicones, oils, associative polyurethane derivatives, associative cellulose derivatives, associative polyvinyllactam derivative,s and associative unsaturated polyacid derivatives.

If present, the at least one conditioner may be present in an amount ranging from 0.0025% to 10% by weight and, for example, from 0.025% to 10% by weight, relative to the total weight of the oxidizing composition.

Needless to say, a person skilled in the art will take care to select this or these optional compound(s) such that the advantageous properties intrinsically associated with the composition(s) disclosed herein are not, or are not substantially, adversely affected by the envisaged addition(s).

In one embodiment, the pH of the oxidizing composition ranges from 2 to 10.

The process using the composition will now be described.

In one embodiment, the process comprises:
a) applying, to wet or dry keratin fibers, at least one composition comprising, in a cosmetically acceptable medium,
   (i) at least one reducing agent and
   (ii) at least one conductive polymer;
b) leaving the at least one composition on the fibers for a time period that is sufficient to shape the fibers;
c) optionally rinsing the fibers and optionally applying at least one oxidizing composition to the keratin fibers;
d) leaving the at least one oxidizing composition on the keratin fibers for a time period that is sufficient to fix the shape of the fibers;
e) optionally rinsing the fibers;
f) washing and rinsing the fibers; and
g) drying the fibers or leaving the fibers to dry.

It should be noted that, during step b), the keratin fibers may be shaped, in other words smoothed out or placed under tension using curlers, before or after the application of the at least one composition, or simultaneously therewith.

The leave-in time period may range from 3 to 30 minutes and, for example, from 5 to 20 minutes.

Moreover, and purely as a guide, steps a) and b) may be performed at a temperature ranging from 15 to 80° C. and, for example, ranging from 20 to 40° C.

Once step b) has been performed, the fibers may be rinsed, for example, with water, and the at least one oxidizing composition may be applied.

It is usually important for the shape given to the fibers to be conserved during this step.

The leave-in time period of the at least one oxidizing composition may range from 3 to 30 minutes and, for example, from 5 to 15 minutes.

In addition, the temperature at which this step may be performed conventionally ranges from 15 to 80° C. and, for example, from 20 to 40° C.

In yet another embodiment, once step d) has been performed, the fibers impregnated with the oxidizing composition may be rinsed, generally with water.

Where appropriate, the keratin fibers may be separated from the means required to keep it under tension, before or after rinsing.

Finally, during step f), the treated fibers are optionally washed, for example, with shampoo, and then rinsed, usually with water, and then, during a step g), the fibers are dried or are left to dry.

Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the disclosed composition. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the present disclosure are approximations, the numerical values set forth in the specific examples are reported as precisely as possible. Any numerical value, however, inherently contain certain errors necessarily resulting from the standard deviation found in their respective testing measurements. The following examples are intended to illustrate the present disclosure without limiting the scope as a result.

### EXAMPLE 1

Synthesis of poly(thiophene-3-acetic acid)

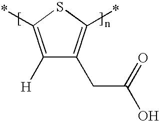

Procedure

Preparation of the polymer: poly(ethyl thiophene-3-acetate)

25 ml of dry chloroform were introduced into a Schlenk tube under argon, the system was degassed and the following reagents were then introduced: 2.5 g of ethyl thiophene-3-acetate (14.7 mmol) 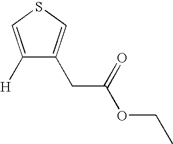
and 1 g of FeCl₃ (6.15 mmol).

The mixture was stirred for 24 hours under argon at 50° C.

The poly(ethyl thiophene-3-acetate) polymer was then precipitated in heptane.

The polymer was then dissolved in a tetrahydrofuran solution.

Infrared characterization:

C═O band: 1719 cm⁻¹; CH₂, CH₃ bands=2979 cm⁻¹, 2934 cm⁻¹ and disappearance of the CH band at 3102 cm⁻¹ present in the monomer.

Hydrolysis of the polymer: poly(ethyl thiophene-3-acetate) to form poly(thiophene-3-acetic acid)

The polymer obtained above was then hydrolysed with an excess of 50 ml of an aqueous sodium hydroxide solution (2N) for 48 hours at 70° C., followed by acidification with concentrated HCl up to the point of precipitation of the product: poly(thiophene-3-acetic acid).

The polymer was then filtered off and washed several times with distilled water in order to remove the traces of catalyst.

Infrared characterization of the Polymer:

C═O band: 1740 cm⁻¹; COO 1580 cm⁻¹; OH (broad band 3000-3500 cm⁻¹)

Neutralization of the poly(thiophene-3-acetic acid) polymer:

The poly(thiophene-3-acetic acid) polymer (2 g) was dissolved in tetrahydrofuran (30 g) and neutralized with a proportion of 1 mol of sodium hydroxide per mole of carboxylic acid.

Water (30 g) was then added.

The tetrahydrofuran was evaporated off.

An aqueous 6% solution of poly(thiophene-3-acetic acid) in the form of a sodium salt was thus obtained.

Formulation 1 comprising the polymer and process using it:

| | |
|---|---|
| Poly(thiophene-3-acetic acid) | 5 g |
| Thioglycolic acid | 3 g |
| Monoethanolamine | pH 8 |
| Ethyl alcohol | 20 g |
| Water qs | 100 g |

Formulation 1 was applied to wet dark hair that had been rolled up beforehand on a roller 9 mm in diameter. After a leave-in time of 20 minutes, an oxidizing lotion containing 8 volumes of aqueous hydrogen peroxide solution brought to pH 3 with citric acid was applied. This lotion was left on for 5 minutes. The hair was then unrolled and the drying process was performed (free drying).

### EXAMPLE 2

Formulation 2 Comprising the Polymer and Process Using it

| | |
|---|---|
| Poly(thiophene-3-acetic acid) | 5 g |
| Cysteine hydrochloride | 7 g |
| Monoethanolamine | pH 9.4 |
| Ethyl alcohol | 20 g |
| Water qs | 100 g |

Formulation 2 was applied to wet dark hair that had been rolled up beforehand on a roller 9 mm in diameter. After a leave-in time of 30 minutes at 45° C., the hair was left to stand for 10 minutes at room temperature. The hair was unrolled and left to stand for 15 minutes. The drying process was then performed (free drying).

What is claimed is:

1. A composition comprising, in a cosmetically acceptable medium,
(a) at least one reducing agent and
(b) at least one conductive polymer.

2. The composition according to claim 1, wherein the at least one conductive polymer comprises at least one repeating unit chosen from:
anilines of formula (I) below: 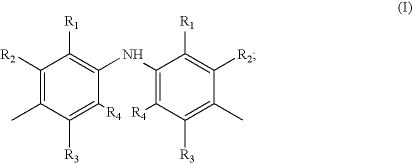
pyrroles of formulae (IIa) and (IIb) below: 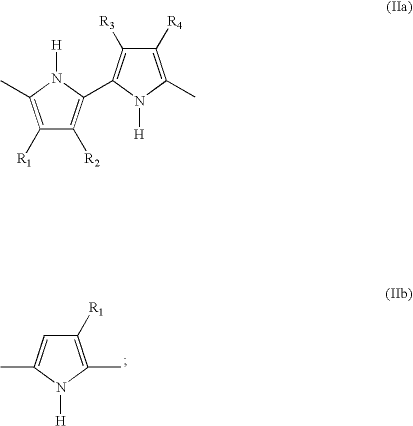
thiophenes and bisthiophenes of formulae (IIIa), (IIIb) and (IIIc) below: 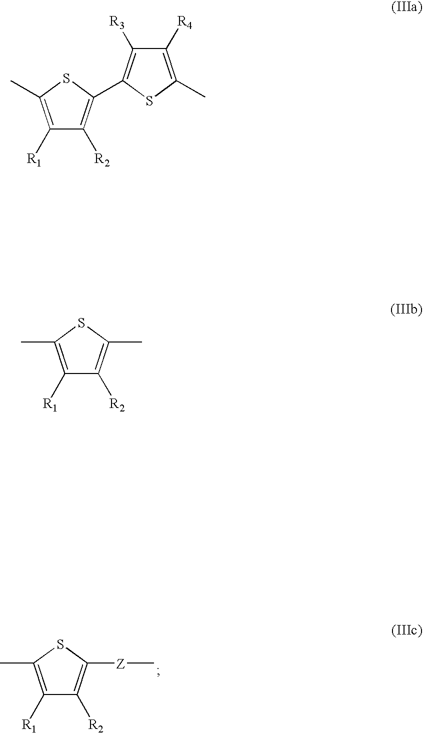
furans of formula (IV) below: 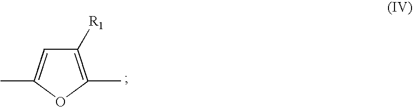
para-phenylene sulfides of formula (V) below: 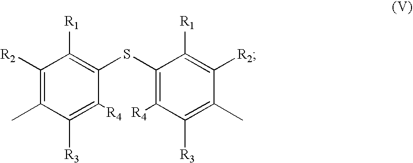
para-phenylenevinylenes of formula (VI) below: 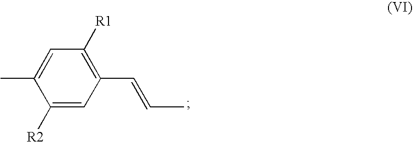
indoles of formula (VII) below: 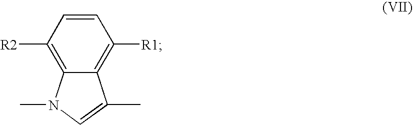
aromatic amides of formulae (VIIIa), (VIIIb), (VIIIc) and (VIIId) below: 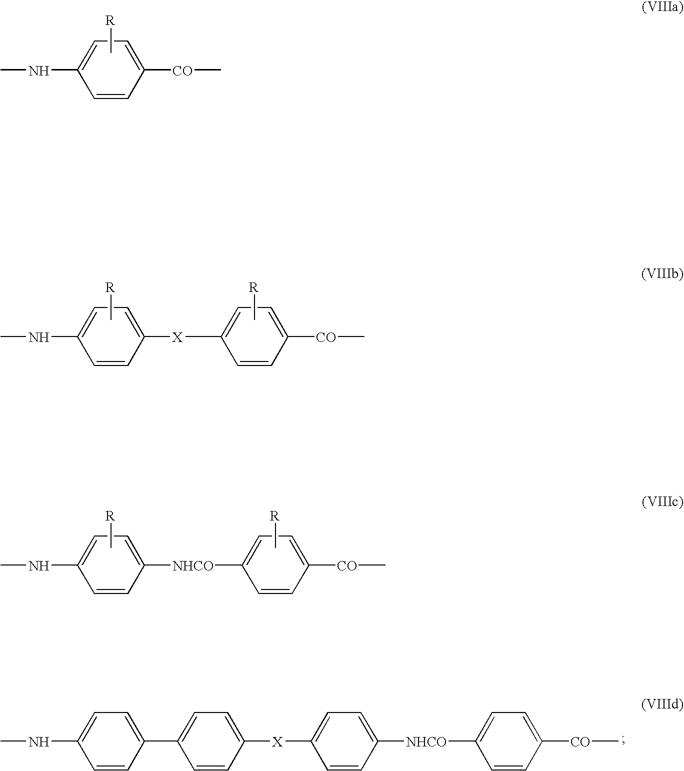
aromatic hydrazides of formulae (IXa), (IXb) and (IXc) below: 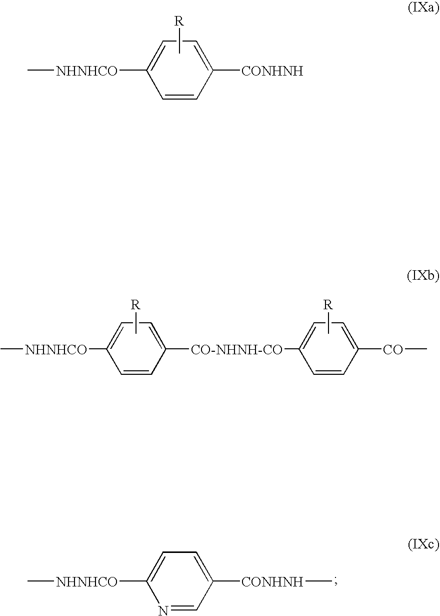
aromatic azomethines of formulae (Xa), (Xb) and (Xc) below: 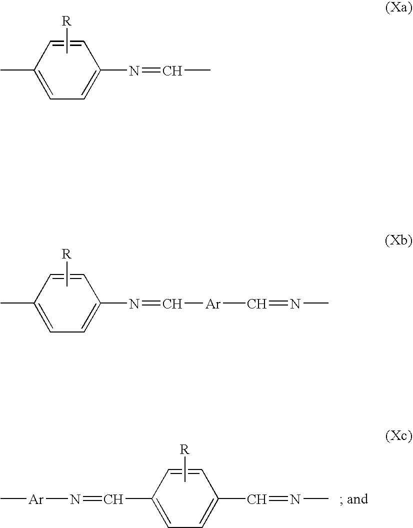
aromatic esters of formulae (XIa), (XIb) and (XIc) below: 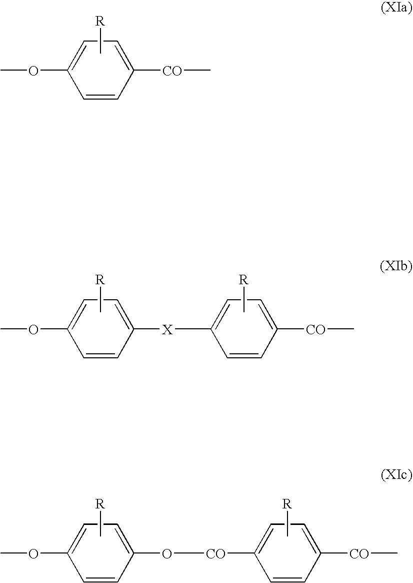
wherein, in formulae (I) to (XI):
R, R₁, R₂, R₃, and R₄, which may be identical or different, are each chosen from a hydrogen atom; —R′, —OR′, —COOR′, and —OCOR′, wherein R′ is chosen from linear and branched C₁-C₂₀ alkyl radicals; halogen atoms; nitro radicals; cyano radicals; cyanoalkyl radicals; solubilizing groups; and solubilizing groups comprising a spacer group that bonds to the ring;
Ar is chosen from radicals comprising at least one radical chosen from monoaromatic and polyaromatic radicals;
X is chosen from —NHCO—, —O—, —S—, —SO₂—, —N═N—, —C(CH₃)₂—, —CH₂—, —CH═CH—, and —CH═N—; and
Z is chosen from —CH═CH— and —C≡C—.

3. The composition according to claim 2, wherein the solubilizing groups are chosen from:
carboxylic (—COOH) radicals and carboxylate (—COO⁻M⁺) radicals, wherein M is chosen from alkali metals, alkaline-earth metals, organic amines, alkanolamines, and amino acid radicals;
sulfonic (—SO₃H) radicals and sulfonate (—SO₃⁻M⁺) radicals, wherein M is chosen from alkali metals, alkaline-earth metals, organic amines, alkanolamines, and amino acid radicals;
primary, secondary, and tertiary amine radicals;
quaternary ammonium radicals;
hydroxyl radicals, and
poly((C₂-C₃)alkylene oxide) radicals.

4. The composition according to claim 3, wherein the quaternary ammonium radicals are chosen from —N(R′)₃⁺Z⁻ groups, wherein Z is chosen from Br, Cl, and (C₁-C₄)alkyl-OSO₃ and R′, which may be identical or different, is chosen from linear and branched C₁ to C₂₀ alkyls, or two of the R's form a heterocycle with the nitrogen to which they are attached.

5. The composition according claim 2, wherein the solubilizing groups are connected to the ring via a spacer group.

6. The composition according to claim 5, wherein the spacer group is chosen from —R″—, —OR″—, —OCOR″—, and —COOR″—, wherein R″ is chosen from linear and branched C₁-C₂₀ alkyl radicals optionally comprising at least one hetero atom.

7. The composition according to claim 2, wherein the radicals R and R₁ to R₄, which may be identical or different, are each chosen from hydrogen, R′, —OR′, —OCOR′—COOR′, wherein R′ is chosen from linear and branched C₁-C₆ alkyl radicals, and from the following neutralized or non-neutralized solubilizing groups: —COOH, —CH₂COOH, —CH₂OH, —(CH₂)₆OH, —(CH₂)₃SO₃H, —O(CH₂)₃SO₃H, —O(CH₂)₃N(CH₂CH₃)₂, —[(CH₂)₂O]ₓCH₂CH₂OH, and —[(CH₂)₂O]ₓCH₂CH₂OCH₃, wherein x is an average number ranging from 0 to 200.

8. The composition according to claim 2, wherein at least one radical, chosen from R and R1 to R4, of the at least one conductive polymer is a solubilizing group.

9. The composition according to claim 2, wherein the at least one conductive polymer comprises at least one solubilizing group per repeating unit.

10. The composition according to claim 2, wherein the solubilizing groups are chosen from carboxylic acid radicals; neutralized carboxylic acid radicals; carboxylic sulfonic acid radicals; neutralized sulfonic acid radicals; tertiary amine radicals; quaternary ammonium radicals, wherein the solubilizing groups are optionally connected to the ring via a spacer group; and salts thereof.

11. The composition according to claim 10, wherein the quaternary ammonium radicals are chosen from N(R′)₃⁺Z⁻ groups, wherein Z is chosen from Br, Cl, and (C₁-C₄)alkyl-OSO₃ and R′, which may be identical or different, is chosen from linear and branched C₁-C₂₀ alkyl radicals.

12. The composition according to claim 10, wherein the spacer group is chosen from C₁-C₂₀ alkyl radicals.

13. The composition according to claim 2, wherein the at least one conductive polymer comprises at least one repeating unit chosen from units of formula (IIIa), (IIIb), and (IIIc), wherein at least one radical chosen from R₁, R₂, R₃, and R₄ of formula (IIIa), and R₁ and R₂ of formulae (IIIb) and (IIIc) is chosen from carboxylic acid solubilizing groups, in neutralized or non-neutralized form, optionally connected to the ring via a spacer group, and wherein the other radical(s) is (are) hydrogen(s).

14. The composition according to claim 13, wherein the spacer group is chosen from linear and branched C₁-C₂₀ alkyl radicals.

15. The composition according to claim 1, wherein the at least one conductive polymer is present in an amount greater than or equal to 0.001% by weight, relative to the total weight of the composition.

16. The composition according to claim 15, wherein the at least one conductive polymer is present in amount less than or equal to 50% by weight, relative to the total weight of the composition.

17. The composition according to claim 15, wherein the at least one conductive polymer is present in an amount ranging from 0.1% to 50% by weight, relative to the total weight of the composition.

18. The composition according to claim 1, wherein the at least one conductive polymer has a conductivity ranging from 1×10⁻⁵ to 5×10⁵ siemens/cm.

19. The composition according to claim 18, wherein the at least one conductive polymer has a conductivity ranging from 1×10⁻³ to 1×10⁵ siemens/cm.

20. The composition according to claim 19, wherein the at least one conductive polymer has a conductivity ranging from 1×10⁻¹ to 1×10⁴ siemens/cm.

21. The composition according to claim 1, wherein the at least one reducing agent is chosen from sulfur-comprising reducing agents and reductones.

22. The composition according to claim 21, wherein the sulfur-comprising reducing agents are chosen from compounds comprising at least one functional group chosen from thiol, sulfide, and sulfite functional groups.

23. The composition according to claim 22, wherein the sulfur-comprising reducing agents are chosen from at least one of thioglycolic acid, β-mercaptoethanol acid, thiolactic acid, alkali metal and alkaline-earth metal salts thereof and esters thereof; cysteine and cysteamine, and derivatives thereof; homocysteine and a salt thereof; mercaptoaldehyde; penicillamine; glutathione; thioglycolate; alkali metal, alkaline-earth metal and ammonium sulfites, bisulfites and hydrosulfites; and cystine.

24. The composition according to claim 21, wherein the reductones are chosen from ascorbic acid and isoascorbic acid, and salts and esters thereof; hydroxypropanedial, 2,3-hydroxy-2-cyclopenten-1-one; and α-ketoglutaric acid.

25. The composition according to claim 21, wherein the at least one reducing agent is present in an amount ranging from 1% to 30% by weight, relative to the total weight of the composition.

26. The composition according to claim 25, wherein the at least one reducing agent is present in an amount ranging from 5% to 20% by weight, relative to the total weight of the composition.

27. The composition according to claim 1, wherein the cosmetically acceptable medium is chosen from water and mixtures of water and at least one cosmetically acceptable organic solvent.

28. The composition according to claim 27, wherein the at least one cosmetically acceptable organic solvent is chosen from C₁-C₄ alcohols, aromatic alcohols, glycols, glycol ethers, polyols, polyethylene glycols, and polypropylene glycol.

29. The composition according to claim 1, further comprising at least one surfactant chosen from nonionic, anionic, cationic, amphoteric, and zwitterionic surfactants.

30. A process for treating keratin fibers comprising,
a) applying, to wet or dry keratin fibers, at least one composition comprising, in a cosmetically acceptable medium,
(i) at least one reducing agent and
(ii) at least one conductive polymer;
b) leaving the at least one composition on the fibers for a time period that is sufficient to shape the fibers;
c) optionally rinsing the fibers and optionally applying at least one oxidizing composition to the keratin fibers;
d) leaving the at least one oxidizing composition on the keratin fibers for a time period that is sufficient to fix the shape of the fibers;
e) optionally rinsing the fibers;
f) washing and rinsing the fibers; and
g) drying the fibers or leaving the fibers to dry.

31. The process according to claim 30, wherein the keratin fibers are human keratin fibers.

32. The process according to claim 31, wherein the human keratin fibers are hair.

33. A method for imparting at least one optical effect on keratin fibers comprising applying to the fibers, at least one composition comprising, in a cosmetically acceptable medium, at least one conductive polymer and at least one reducing agent.

34. The method according to claim 33, wherein the at least one optical effect is sheen.

35. A multi-compartment kit comprising,
at least one compartment comprising, at least one composition comprising, in a cosmetically acceptable medium, at least one conductive polymer and at least one reducing agent and
at least one other compartment comprising at least one oxidizing composition.
